(19) United States
(12) Patent Application Publication
(10) Pub. No.: US 2009/0012373 A1
(43) Pub. Date: Jan. 8, 2009

(54) System and Method for Providing Health Care Services

(75) Inventors: Katariina Elisabet Raij, Espoo (FI); Arja Leena Piirainen, Helsinki (FI); Sinikka Lehto Paula, Espoo (FI); Pasi Nissinen, Espoo (FI); Jussi Tapio Kokkila, Espoo (FI)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Laurea ammattikorkeakoulu Oy

(21) Appl. No.: 12/062,840

(22) Filed: Apr. 4, 2008

Related U.S. Application Data
(60) Provisional application No. 60/910,531, filed on Apr. 6, 2007.

(51) Int. Cl. A61B 5/00; G06Q 50/00

(52) U.S. Cl. 600/300; 705/2

(57) ABSTRACT

A system and method are provided for providing health care. A plurality of virtual presence devices is arranged to a corresponding plurality of customer residences, each comprising a display device; an input device having a user interface permitting selection of an available service; a camera and microphone for interactive audiovisual communication; and a computer device responsive to one of the user interface, the camera, and the microphone, to facilitate the interactive audiovisual communication over an IPTV data network. A health care contact center is in communication with the virtual presence devices, and comprises an IPTV transceiver device configured to interact and audiovisually communicate with the virtual presence devices; and a processing device for receiving one of a contact request, patient data, and a telemedicine measurement from any of the virtual presence devices, and to form an audiovisual memo therefrom for facilitating flow of patient communication to consulting personnel.

### CROSS-REFERENCE TO RELATED APPLICATIONS

This application claims the benefit of U.S. Provisional Application No. 60/910,531, filed Apr. 6, 2007, which is incorporated by reference herein in its entirety.

### BACKGROUND OF THE INVENTION

1. Field of the Invention

Embodiments of the invention relate to a system (Care TV) and method for caring for persons, such as elderly persons, in their home using telecommunications and interactive multimedia.

2. Description of Related Art

Elderly persons, particularly those seeking to live independently as long as possible, may often have a difficult time pursuing appropriate health care services. One possible reason is that it may be difficult for that elderly person to travel outside the home to the appropriate health care service provider. In such cases, it may also be impractical for such needed health care services to be brought to the home of the person, or such services may be unavailable to the person in their home, outside of emergency services.

Elderly persons living independently may also suffer from a lack of socialization, which may also lead to health issues. In some instances, one of the only forms of socialization for those persons may lie in a television set found in the home.

Thus, there exists a need for a system and method for providing health care services into a home, particularly a home occupied by elderly persons. Such a system and method should also desirably be at least partially interactive, so as to allow the health care provider to remotely evaluate a person in the home, or allow the person in the home to request health care services. In addition, such a system and method should also desirably permit selective third party participation, for example, when a second opinion is necessary or desirable. It may also be desirable for such a system and method to provide a socialization function or otherwise provide useful information to the person in the home.

### SUMMARY OF THE INVENTION

The above and other needs are met by the present invention which, according to one aspect, provides a system for providing health care using virtual presence. Such a system comprises a plurality of virtual presence devices arranged to a corresponding plurality of residences of health care customers. Each virtual presence device further comprises a display device configured to display video data. An input device having a user interface is configured to control the display device and permit a selection of a service available through the display device via the user interface. A camera and a microphone facilitate interactive audiovisual communication. A computer device is operably engaged with the display device, the input device, the camera, and the microphone, and is configured to be responsive to at least one of the user interface, the camera, and the microphone, to facilitate the interactive audiovisual communication over an IPTV data network. A health care contact center is in communication with the virtual presence devices, and comprising an IPTV transceiver device configured to be capable of interactively and audiovisually communicating with the virtual presence devices. A processing device is in communication with the IPTV transceiver device and is configured to receive at least one of a contact request, patient data, and a telemedicine measurement from any of the virtual presence devices, and to form an audiovisual memo therefrom. The audiovisual memo is thus adapted to facilitate flow of patient communication to consulting personnel.

Another aspect of the present invention comprises a method for providing health care services. Such a method involves providing a plurality of virtual presence devices to a corresponding plurality of residences of health care customers, wherein each virtual presence device further comprises a display device configured to display video data, and an input device having a user interface and configured to control the display device and permit a selection of a service available through the display device via the user interface. A camera and a microphone facilitate interactive audiovisual communication. A computer device is operably engaged with the display device, the input device, the camera, and the microphone, and is configured to be responsive to at least one of the user interface, the camera, and the microphone, to facilitate the interactive audiovisual communication over an IPTV data network. A health care contact center is arranged in communication with the virtual presence devices, wherein the health care contact center comprises a database including patient health care data, and an IPTV transceiver device configured to be capable of interactively and audiovisually communicating with the virtual presence devices. A processing device is in communication with the IPTV transceiver device and is configured to receive at least one of a contact request, patient data, and a telemedicine measurement from any of the virtual presence devices, and to access the database to determine the patient health care data, and to form an audiovisual memo therefrom. The audiovisual memo is thus adapted to facilitate flow of patient communication and data to consulting personnel.

In another aspect, such a method may further comprise receiving one of patient data and the telemedicine measurement at the processing device from a patient information device in communication with the virtual presence device and interacting with a patient. Yet another aspect contemplates providing a multiparty audiovisual communication device operably engaged between the virtual presence devices and the health care contact center and adapted to permit a third party to participate in audiovisual communication with any of the virtual presence devices.

Aspects of the present invention thus provide significant advantages as otherwise detailed herein.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIGS. 1 and 2 are schematics of a system for providing health care services, according to one embodiment of the present invention.

### DETAILED DESCRIPTION OF THE INVENTION

The present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

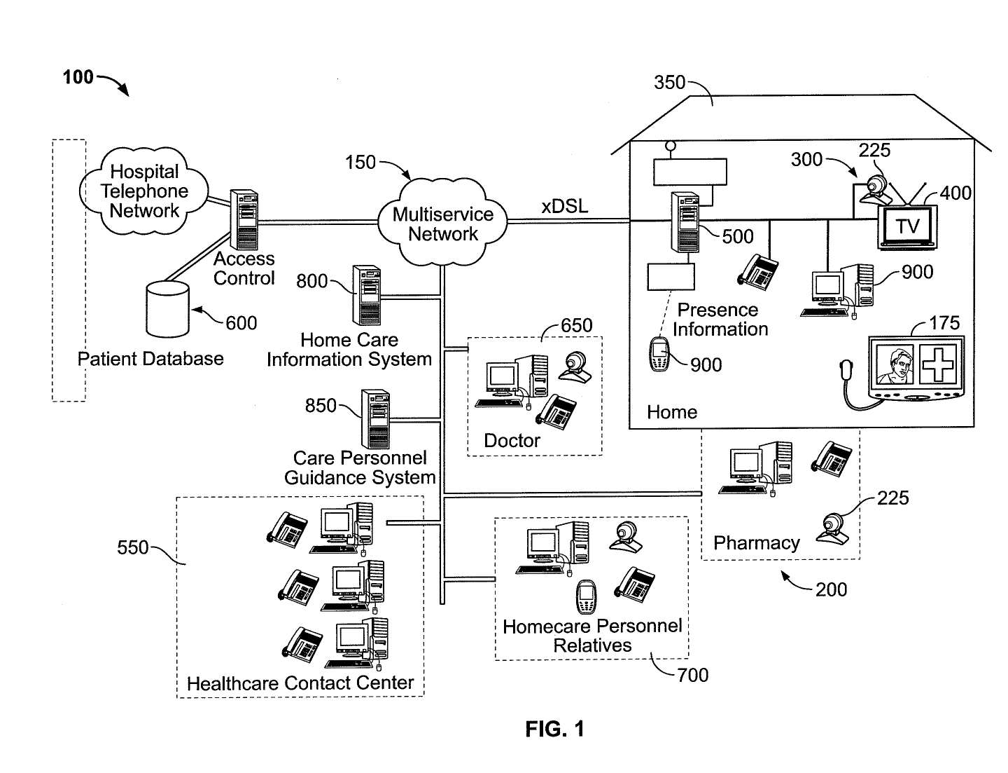

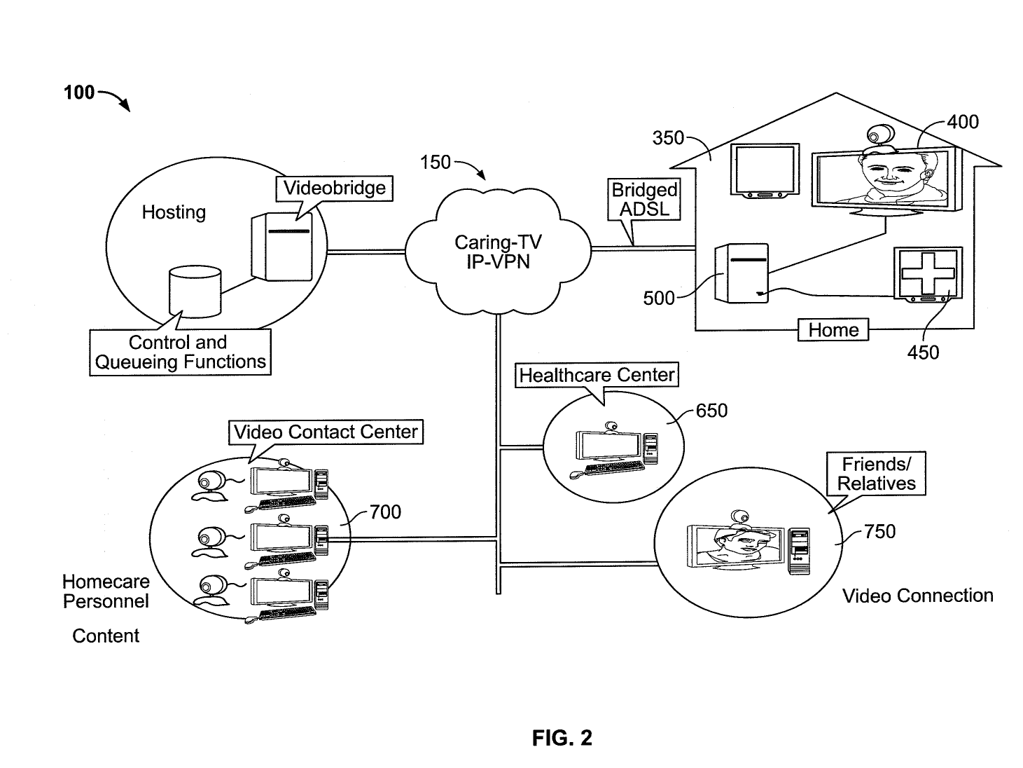

As shown in FIGS. 1 and 2, embodiments of the system 100 provide service through a data communication network marked as Multiservice Network 150. The network 150 preferably comprises a fast backbone with quality of service control and multicast. The network 150 uses internet protocols, with at least some of the communications of the system being encrypted with public key encryption methods.

Pharmacy 200 is an example of a service provider that may join the system 100 and that may use the system 100 as a business media. Other examples may be a food store, a church, a travel agency, library etc. The shop or store may have a wireless camera 225 set for the shopkeeper to collect the goods “online” for the delivery. The camera 225 is used to see the goods and negotiating about the order.

The network 150 may replace the normal cable TV and even telephone network. In that case the video content and TV channels may be multicast in the network 150 and the network 150 may also be used for IP telephony. Some embodiments of the system 100 may be directed to elderly people, which may require particular attention to the use interface. Therefore, the user interface 175 may be “old fashioned” and simple, in some instances hiding the complicated system 100 and processes behind the system 100. The system 100 may use a closed VPN-type network for communications and the virtual presence device 300 (also referred to herein as a “Care TV set”) at a particular site (i.e., customer's residence or house 350) may thus have a simple user interface 175. In one aspect, the virtual presence device 300 is configured to substantially prevent the local installation of software, thereby providing a more secure device.

The virtual presence device 300 in the home 350 may include a display device 400 such as a television (TV), and an input device 450 having a user interface (i.e., a separate touch-screen display). Also a touch-screen enabled TV is possible as another display device 400/input device 450. One particular aspect involves a tablet-type personal computer (PC) with a touch-screen as one aspect of an input device 450/user interface. As such, a user interface with large and clear buttons on the touch screen can be devised, and the TV 400 is adequately large and high quality for video use to give a feel of presence. The display device 400 and input device 450 can be any adequately large and TV-like device that also works as a TV 400. The virtual presence device(s) 300 are connected to the system 100, for example, by an internet connection like xDSL, or any other suitable connection media such as 3G for wireless connections, or optical fibers, via an appropriate computer device 500.

The system 100 and service provided thereby may also allow many people to keep social contacts 750 through the network. For example, instead of only video telephony via internet, the system 100 may be configured to allow multicast and/or interactive or bi-directional connections between multiple users. The system 100 may also be configured to imitate a classroom by a changeable view combined from several streams in each terminal or TV. That is, a broadcast center arranging a lesson or meeting may see a large group of users simultaneously from a multiplicity of monitors or split monitor(s), while the center that arranges broadcast may select or combine several streams from which the viewer may select. The other channels may be presented in a picture-in-picture view for facilitating an easy selection of a picture. The method makes it easy to form ad hoc clubs of users or arrange a meeting. The selection of video streams may be done by the touch screen panel 450 present in the home 350.

Health care contact center 550 works as a first contact point for the elderly people (or other service customers) or those caring for close relatives. The contact center 550 can make real time connections to multiple parties and they may connect the call to a doctor, to a hospital, or to a paramedic. The connection may be a multiparty connection, so that the contact center operator can help the customer to explain his/her needs to a doctor. The contact center 550 may help the customer for any problem, for example the center 550 may take a voice-telephone call and help the customer in ordering some service. The video connection helps the contact center operator's understanding and communication, when three or more persons are on the same telephone conversation. The customer can show the problem to the operator, and the operator may help by asking for help or directing services.

The contact center 550 can use the video connection for analyzing the situation and ask for further help if needed. If, for example, the contact center operator is a nurse, she/he can ask the preliminary questions and preferably use also telemetry for measuring for example the blood pressure, the glucose value, the pulse rate, etc. Also the contact center 550 can determine the “patient file” from a database 600 before passing the case further to the doctor 650. This kind of workflow management enhances the productivity of the work. The system 100 may be connected to a wrist clinic (or presence information module 900) for interacting with the patient/customer to provide medical monitoring functions like: heart rate, ECG, blood pressure, heart rhythm regularity, respiratory rate, Oxygen saturation (SpO2), and body temperature. These kinds of devices are readily available.

For helping the work flow management the patients or the customers have identification information and the access control of the patient data is restricted to co-operation of the patient. The patient data visible to the workers of the contact center 550 includes at least the contact information of the patient's own doctor and other persons who may be helpful. The contact center person may record a video memo together with the patient information/data and send it to the doctor 650 or other health care personnel 700. The doctor gets the video memo, together with the measurement data and other patient information, if available. The doctor's time is saved for most important cases, and the work can be arranged in a pleasant way, as the doctor can see the patient's video memo before contacting him/her. The doctor may handle the video memos without reserved times. The video memo helps remembering the patient and the non-verbal communication may be very important especially with handicapped persons, elderly persons or with children. The pilot test of the system 100 shows that the elderly persons find it very easy to use the TV for video calls, the feel of presence is important also for keeping the social contacts more alive. The system 100 enables a nurse to keep contact to several patients like they were in a virtual care home. This makes the contacts more frequently than physical visits could be with the same economic contribution. This enables the elderly people to live in their homes much longer instead of early institutionalization.

The system and method according invention has proven to be activating for social contacts and it has proven to help the elderly people to maintain their ability to keep themselves fit both physically and mentally. Interactive connection to the instructor and possibly to the rest of the group makes the exercise programs more motivating. The system 100 helps the healthcare organizations and partner organizations to co-operate more effectively.

A pharmacy 200 is an example of a service provider connected to the system 100. The service provider may get access to necessary parts of the database 600 or the data may be sent as e-mail or other suitable manner according to the case. The pharmacy 200 may be able to connect to the medical databases and get prescriptions in electronic form. In that case the pharmacy 200 may need to have a user account in the system 100, as the prescriptions need to be verified and electronically stamped and stored to the database. If the pharmacy 200 has only a standard type of video conferencing device, the system 100 may need a separate gateway for passing the prescription to the pharmacy and “stamping” the used prescription in the database. Other service providers may be, for example, a food store, a library, a travel agency, or practically any type of business or service. The service provider may have a wireless camera set for collecting the goods on-line with the customer.

The user interface of the elderly person or other customer is preferably a TV 400. The system 100 may thus comprise a set of IPTV channels of video-on-demand services. The system 100 may replace the normal cable TV of a hospital or an old-age home, or the system 100 may comprise an integrated receiver for easy connection to traditional TV channels. The TV 400 is controlled by touch screen or by an ordinary remote controller as the input device 450. A touch screen is one preferable media, as it allows simple buttons and menus. The user interface is preferably a web-page. Most of the software is in the servers, and may thus make the updating and maintenance of the system easy. The touch screen may be on TV or on a separate small display. The touch screen panel may be a consumer electronics product, like a game console, internet pad, PDA, or mobile phone. The touch screen may be on a separate computer or only a screen connected to the computer that also provides the TV-image and IPTV-channels. A separate computer may be easily wireless, or a screen may need a cable or other wireline connection.

The terminal or user may need an encryption key set for identification, in form of for example a SIM card, pass word, preprogrammed ID number, or biometric recognition. As the terminal may be used for economical transactions, security needs to be addressed. Face and voice recognition is one moderately simple way, and can be made in the server so that the patient may use any terminal without need of remembering any password. The user interface may be configurable and personal. That makes it possible to use any terminal with one's own personal settings.

The camera 225 may be used for surveillance locally or with the help of a server. The use of picture recognition with the camera 225 may be combined with use of life monitoring wristband or strap (presence information module 900) connected to the patient. The needed technology is available at least from Finnish companies Polar, Suunto and IST.

A home care information system 800 stores health information. The nurse or doctor may use the portable terminal or home terminal with keyboard to feed information to the home care information system 800 during a house call. The patient database 600 may be combined with the home care information system 800 or the hospital and home care may have access to systems of each other. The servers are presented as functional entities, they may be combined or situated in various ways. The care personnel guidance system 850 is guiding the workflow and giving general instructions. For example the working time management of the care personnel may be done by care personnel guidance system 850.

The computer device 500 in the home 350, illustrated under the TV 400, is configured to collect the patient measurement results, and transmitting the same as necessary. It also performs surveillance, if needed. The computer device 500 may be an integral part of the touch panel or a separate computer. The presence information module 900 is, for example, a Bluetooth tagged mobile phone, a pulse measuring wireless strap, a motion detector, or any other device that can be used for monitoring the well being and activity of the patient/customer in the home 350. The system 100 may be used also for watching pets while the home owner is absent. The system 100 may also comprise a microphone and sound detection, or the wrist band with emergency alarm button.

The home computer or the server 500 may remind the patient of missing values/patient information and/or remind the patient to take a medication. The system 100 can also provide an alarm in response to abnormal values and automatically ask the doctor or nurse to analyze the values.

The doctor can access the home measurement data via the system 100 Also, the health care contact center 550, the patient, and/or the patient's homecare personnel may leave a video memo with the doctor, or e-mail and ask for contact from the doctor. The home system may have a very simple user interface for reserving the appointment time, either for telemedicine services or for visit to the doctor. Calling the doctor may be done only by automated appointment, so that the doctor always accepts the call and calls back. Typically, the health care contact center 550 handles the appointments and helps the users to get the right kind of help, also asking the preliminary questions of the patient and estimating the urgency of help needed. The nurse or doctor automatically has all the collected information of home measured values when contacting the patient via the system 100.

The system 100 may use a UMTS phone to access at least part of the services everywhere. In that case the mobile terminal may contact the home computer device 500, the home care information server 800, or to the IPTV services.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A system for providing health care using virtual presence, comprising:
a plurality of virtual presence devices arranged to a corresponding plurality of residences of health care customers, each virtual presence device further comprising:
a display device configured to display video data;
an input device having a user interface and configured to control the display device and permit a selection of a service available through the display device via the user interface;
a camera and a microphone for facilitating interactive audiovisual communication; and
a computer device operably engaged with the display device, the input device, the camera, and the microphone, the computer device being configured to be responsive to at least one of the user interface, the camera, and the microphone, and to facilitate the interactive audiovisual communication over an IPTV data network; and
a health care contact center in communication with the virtual presence devices, and comprising:
an IPTV transceiver device configured to be capable of interactively and audiovisually communicating with the virtual presence devices; and
a processing device in communication with the IPTV transceiver device and configured to receive at least one of a contact request, patient data, and a telemedicine measurement from any of the virtual presence devices, and to form an audiovisual memo therefrom, the audiovisual memo being adapted to facilitate flow of patient communication to consulting personnel.

2. A system for providing health care according claim 1, wherein the virtual presence device further comprises a medical metering device.

3. A system for providing health care according claim 1, further comprising a multiparty audiovisual communication device operably engaged between the virtual presence devices and the health care contact center and adapted to permit a third party to participate in audiovisual communication therebetween.

4. A system for providing health care according claim 1, wherein the processing device is further configured to facilitate audiovisual communication, via the IPTV transceiver device, of a virtual lesson to any of the virtual presence devices for broadcast to a patient via the display device.

5. A system for providing health care according claim 1, further comprising an audiovisual communication gateway for selectively receiving communication from one of a public Internet and a cellular telecommunication network and directing the communication to one of the health care contact center and any of the virtual presence devices.

6. A system for providing health care according claim 1, wherein the processor device further comprises at least one of a prescription module and a medication management module capable of communicating with any of the virtual presence devices via the IPTV transceiver device.

7. A system for providing health care according claim 1, wherein at least one of the virtual presence devices further comprises a patient information device in communication therewith.

8. A method for providing health care services, comprising:
providing a plurality of virtual presence devices to a corresponding plurality of residences of health care customers, each virtual presence device further comprising:
a display device configured to display video data;
an input device having a user interface and configured to control the display device and permit a selection of a service available through the display device via the user interface;
a camera and a microphone for facilitating interactive audiovisual communication; and
a computer device operably engaged with the display device, the input device, the camera, and the microphone, the computer device being configured to be responsive to at least one of the user interface, the camera, and the microphone, and to facilitate the interactive audiovisual communication over an IPTV data network; and
arranging a health care contact center in communication with the virtual presence devices, the health care contact center comprising:
a database including patient health care data;
an IPTV transceiver device configured to be capable of interactively and audiovisually communicating with the virtual presence devices; and
a processing device in communication with the IPTV transceiver device and configured to receive at least one of a contact request, patient data, and a telemedicine measurement from any of the virtual presence devices, and to access the database to determine the patient health care data, and to form an audiovisual memo therefrom, the audiovisual memo being adapted to facilitate flow of patient communication and data to consulting personnel.

9. A method for providing health care services, comprising:
providing a plurality of virtual presence devices to a corresponding plurality of residences of health care customers, each virtual presence device further comprising:
a display device configured to display video data;
an input device having a user interface and configured to control the display device and permit a selection of a service available through the display device via the user interface;
a camera and a microphone for facilitating interactive audiovisual communication; and
a computer device operably engaged with the display device, the input device, the camera, and the microphone, the computer device being configured to be responsive to at least one of the user interface, the camera, and the microphone, and to facilitate the interactive audiovisual communication over an IPTV data network;
arranging a health care contact center in communication with the virtual presence devices, the health care contact center comprising:
a database including patient health care data;
an IPTV transceiver device configured to be capable of interactively and audiovisually communicating with the virtual presence devices; and
a processing device in communication with the IPTV transceiver device and configured to receive at least one of a contact request, patient data, and a telemedicine measurement from any of the virtual presence devices, and to access the database to determine the patient health care data, and to form an audiovisual memo therefrom, the audiovisual memo being adapted to facilitate flow of patient communication and data to consulting personnel; and
receiving one of patient data and the telemedicine measurement at the processing device from a patient information device in communication with the virtual presence device and interacting with a patient.

10. A method for providing health care services, comprising:
providing a plurality of virtual presence devices to a corresponding plurality of residences of health care customers, each virtual presence device further comprising:
a display device configured to display video data;
an input device having a user interface and configured to control the display device and permit a selection of a service available through the display device via the user interface;
a camera and a microphone for facilitating interactive audiovisual communication; and
a computer device operably engaged with the display device, the input device, the camera, and the microphone, the computer device being configured to be responsive to at least one of the user interface, the camera, and the microphone, and to facilitate the interactive audiovisual communication over an IPTV data network;
arranging a health care contact center in communication with the virtual presence devices, the health care contact center comprising:
a database including patient health care data;
an IPTV transceiver device configured to be capable of interactively and audiovisually communicating with the virtual presence devices; and
a processing device in communication with the IPTV transceiver device and configured to receive at least one of a contact request, patient data, and a telemedicine measurement from any of the virtual presence devices, and to access the database to determine the patient health care data, and to form an audiovisual memo therefrom, the audiovisual memo being adapted to facilitate flow of patient communication and data to consulting personnel; and
providing a multiparty audiovisual communication device operably engaged between the virtual presence devices and the health care contact center and adapted to permit a third party to participate in audiovisual communication with any of the virtual presence devices.
